(19) United States
(12) Patent Application Publication
(10) Pub. No.: US 2024/0148306 A1
(43) Pub. Date: May 9, 2024

(54) METHOD FOR SELECTING ELECTROPHYSIOLOGICAL DESCRIPTORS

(71) Applicants: UNIVERSITE DE BORDEAUX, BORDEAUX (FR); CENTRE HOSPITALIER UNIVERSITAIRE DE BORDEAUX, TALENCE (FR); FONDATION BORDEAUX UNIVERSITE, BORDEAUX (FR)

(72) Inventors: Laura BEAR, TALENCE (FR); Olivier BERNUS, LACANAU DE MOIS (FR); Rémi DUBOIS, MERIGNAC (FR); Michel HAISSAGUERRE, TALENCE (FR); Nolwenn TAN, LIBOURNE (FR)

(21) Appl. No.: 18/550,630

(22) PCT Filed: Mar. 25, 2022
(86) PCT No.: PCT/EP2022/057991

§ 371 Date: Sep. 14, 2023

(30) Foreign Application Priority Data
Mar. 25, 2021 (FR) FR2103047

(51) Int. Cl. A61B 5/349; A61B 5/282; G16H 20/30; G16H 50/70

(52) U.S. Cl. CPC A61B 5/349; G16H 20/30; G16H 50/70; A61B 5/282

(57) ABSTRACT

A method for selecting a subset of electrophysiological descriptors from a set of electrophysiological descriptors, includes procedures for estimating values of a set of electrophysiological descriptors and selecting a subset of descriptors as a function notably of quantifications of proximity factors.

### FIELD OF THE INVENTION

The field of the invention relates to methods and devices for selecting a set of electrophysiological descriptors linked to an individual's cardiac activity. More particularly, the field of the invention relates to methods implemented by means of surface electrodes recording signals used to detect a representative cardiac electrical activity.

### PRIOR ART

Currently, there are means for analyzing signals from surface electrodes in order to identify an electrical activity of the myocardium. This analysis makes it possible to prevent certain cardiac pathologies by identifying singular electrical activities within the myocardium.

These means, even though very reliable, only focus on a limited number of cardiac activity indicators derived from the analysis of cardiac electrical signals. For example, the duration of the QRS is frequently used, designating the duration of ventricular depolarization, as an indicator of cardiac pathologies. This measurement is a very good indicator for detecting certain singular activities of the heart, but this measurement alone is only an outline of certain cardiac characteristics. For example, it is possible for a patient with a given pathology that the measurement of QRS duration may be representative of an electrophysiological singularity. However, this measurement may prove to be insufficient to characterize certain electrophysiological activities of an individual, notably an electrophysiological activity that can be used or corroborated with other variables to anticipate a cardiac risk.

Thus, by focusing on few parameters, it is common not to detect characteristic electrophysiological activities.

Currently known methods have the drawback, by focusing on a very limited number of indicators, of offering only a very limited view of a patient's cardiac activity.

To date, existing solutions aim to confirm or detect the presence of a singular electrical activity. However, this activity usually remains at a low level or drowned in noise or even masked by a larger electrical pattern such as a QRS complex. No solution makes it possible, from a plurality of surface electrodes, to determine the characteristics of a source of a singular electrical activity or even to know, from a wide range of electrophysiological variables of a patient, which are likely to form a corpus of singularities that can be used for medical purposes or to prevent a cardiac risk.

The invention therefore aims to propose a method for selecting parameters for measuring the cardiac activity of a patient that addresses the aforementioned drawbacks.

### SUMMARY OF THE INVENTION

According to one aspect, the invention relates to a method for selecting a subset of first electrophysiological descriptors characteristic of a characteristic cardiac electrical activity from a set of first predefined descriptors. Each first electrophysiological descriptor is associated with at least one channel, one signal type, one signal marker and one statistical modality for calculation. The method comprises a recording of a plurality of electrical activities defining said channels and comprises:
Estimation, for a first set of patients not having a predefined condition, of values of each descriptor of the set of predefined descriptors;
Estimation, for a second set of patients having the predefined condition, of values of each descriptor of the set of predefined descriptors;
Generation of a first vector characteristic of the condition of each patient of the first and second sets of patients in which each component corresponds to a condition relative to the predefined condition;
Generation of a descriptor vector for each descriptor in a metric space in which each component corresponds to the value of the descriptor for each patient;
First quantification for each descriptor of a first proximity factor between the values of the components of the first characteristic vector and the values of the components of the descriptor vector;
First selection and inclusion in the subset of at least one descriptor having optimal quantified proximity factor values;
Second quantification:
   of a second proximity factor between the values of the components of each descriptor not selected during the first selection step and the values of the components of the descriptor vector(s) selected during the first selection step; and
   a third proximity factor between the values of the components of each descriptor not selected during the first selection step and the characteristic vector component values;
Second selection and inclusion in the subset of at least one new descriptor as a function of the value of the second proximity factor and the value of the third proximity factor quantified during the step of second quantification.

One advantage of the invention is to propose a method for selecting a subset of different electrophysiological descriptors to characterize a cardiac activity. The selection method according to the invention makes it possible to select a subset of electrophysiological descriptors that is relevant to discriminate between two different populations, from a set of physiological descriptors. Thus, it is possible to select from a large number of descriptors that take into account different signal types, different signal markers and various statistical modalities for calculation, a relevant subset of descriptors. Furthermore, since the method according to the invention comprises a second selection step taking into account the third proximity factor, a set of electrophysiological descriptors that are not redundant with each other is obtained to characterize said cardiac activity. The selection method according to the invention therefore makes it possible to obtain an effective and non-redundant subset for the discrimination of two populations.

According to one embodiment, at least one first descriptor of the subset is associated with a statistical modality different from that of another first descriptor of the subset and a signal marker different from that of the other first descriptor. This provision makes it possible to obtain a sub-assembly which comprises several descriptors bearing different physiological information from each other.

The different steps of the method according to the invention may be implemented by calculation means such as calculators. The latter may be those of an electronic board of dedicated equipment or those of a remote computer or data server.

According to one embodiment, the steps of second quantification and second selection are reproduced from the descriptors not selected previously until a predefined number of selected descriptors is obtained. This provision makes it possible to build the subset of descriptors iteratively by each time taking descriptors bearing information different from those borne by the descriptors already selected.

According to one embodiment, the quantification of the first proximity factor and/or the second proximity factor, and/or the third proximity factor is a correlation calculation. A correlation calculation is an effective way of estimating the proximity between two vectors. For example, the correlation is made between vectors defined in the metric space.

According to one embodiment, the step of second quantification comprises a step of projection, in a first plane orthogonal to the descriptor vector(s) associated with the last selected descriptors, of each unselected descriptor vector and the characteristic vector; in that the quantification of the third proximity factor and performed from the projected vector projection components; and in that the second selection is made as a function of the third proximity factor only. According to this provision, the projection in the plane orthogonal to the selected descriptor vector makes it possible to remove the descriptor vectors very close to the selected descriptor vector. This provision therefore makes it possible to select one or more descriptor vectors that bear different information.

According to another embodiment, the step of second quantification may be implemented by another operator than the projection. An operator making it possible to define a distance in the metric space of vectors may be used to calculate one of the proximity factors, notably the first proximity factor, the second proximity factor, and the third proximity factor. According to one embodiment, different operators may be configured so as to calculate the different proximity factors. It is understood in the present invention that a proximity factor is comprised with respect to a distance defined in the metric space. Thus, certain distances may have advantageous properties depending on the proximity factor that is measured. Certain operators favor certain dimensions of vectors during quantification, others make it possible to favor a measurement of approximation or proximity of vectors with each other, while others favor the distance, the orthogonalization or the difference of vectors between them. Thus, the projection of two vectors makes it possible to obtain a quantification of a proximity factor in a subspace, however other methods, such as calculating the quadratic error between two vectors, may be used to define a distance for the purpose of quantifying a proximity factor.

According to one embodiment, the predefined number of selected descriptors is determined by testing the effectiveness of a set of first “n” selected descriptors to characterize a given electrical activity, “n” corresponding to the number of descriptors taken from the selected descriptors, starting from the first to the nth selected descriptor, and incrementing the value of n by steps of 1. This provision makes it possible to calibrate the size of the subset of descriptors to have the subset with the greatest effectiveness.

According to one embodiment, the set comprises at least one second geographical descriptor associated with several channels and several geographical groups, each geographical group being formed by a central channel and the at least four channels close to the central channel, the value of the electrophysiological descriptor being determined:
By comparing the value, for each geographical group, of the measurement of each channel according to the signal type and the signal marker selected with at least one geographical threshold value specific to said electrophysiological descriptor and said channel; and
By counting the number of geographical groups for which the value of at least three channels exceeds its own geographical threshold value.

According to this provision, in addition to the first electrophysiological descriptors, second descriptors are used which take into account geographical groups. The addition of this other type of descriptors makes it possible to take into account the concentration of singular measured values and enriches the subset of selected descriptors.

According to one embodiment, for each first descriptor, the at least one channel is derived from a predefined area on the body of the patient is chosen between:
An upper right area of the torso;
An upper left area of the torso;
A lower right area of the torso;
A lower left area of the torso; and
The entire torso of the patient.

This provision makes it possible to have descriptors that take into account the geographical location on the patient's body of the measurements taken. Thus, there is a variety of descriptors that take into account several types of information.

According to one embodiment, for each descriptor, the signal type analyzed is chosen between:
A unipolar signal taken between an electrode of the chosen body area and a reference electrode;
A vertical bipolar signal taken between two electrodes of the given area, one of the two electrodes being offset along a vertical line with respect to the other electrode;
A horizontal bipolar signal taken between two electrodes of the given area, one of the two electrodes being offset along a horizontal line with respect to the other electrode; and
A Laplacian signal estimated by subtracting from the potential of a central electrode the average tension of the eight electrodes directly adjacent to said central electrode.

This provision makes it possible to take into account several types of signals and therefore to have descriptors that take into account several types of signal measurement. Thus, the descriptors take into account several types of information and report more faithfully on the condition of said patient.

According to one embodiment, the reference electrode is an electrode arranged on the surface of an upper limb or lower limb of the patient. This provision makes it possible to measure a reference potential.

According to one embodiment, a plurality of reference electrodes is arranged on the surface of the patient's lower or upper limb(s). This provision makes it possible to obtain a reference potential with high precision.

According to one embodiment, for at least one descriptor, the signal marker is the voltage measurement of an averaged signal. The averaging of the signal makes it possible to obtain a stable measurement of said voltage.

According to one embodiment, for at least one descriptor, the signal marker is the measurement, on the averaged and filtered signal between 40 and 250 Hertz, of the duration of depolarization of the ventricles or fragmentation of the signal during depolarization of the ventricles. The duration of ventricular depolarization is a very representative measurement of cardiac activity.

According to one embodiment, for at least one descriptor, the signal marker is the measurement on the discrete wavelet decomposition of the signal:
Of the energy of the sum of the wavelets;
Of the Kurtosis;
Of the Fisher asymmetry coefficient; or
Of the number of local minima.

This provision enables different types of signals all bearing different information enriching the selected subset to be taken into account.

According to one embodiment, for at least one descriptor, the signal marker is the measurement on the continuous wavelet decomposition of the signal of the number of local maxima chains. This measurement is a measurement that makes it possible to report a singular cardiac activity.

According to one embodiment, for at least one descriptor, the signal marker is the measurement on the wavelet taken between 256 and 512 Hertz of the signal:
Of the Kurtosis; or
Of the number of areas with reduced amplitudes.

These measurements make it possible to report measurements on the curve such as the flattening thereof.

According to one embodiment, for at least one descriptor, the signal marker is the measurement on the wavelet taken between 128 and 256 Hertz of the signal:
Of the Kurtosis;
Of the number of areas of reduced amplitude; or
Of the RMS (Root Mean Square).

These measurements make it possible to report measurements on the curve such as the flattening thereof.

According to one embodiment, for at least one descriptor, the signal marker is the measurement on the wavelet taken between 64 and 128 Hertz of the RMS (Root Mean Square). This measurement is used to report the patient's cardiac activity.

According to one embodiment, for each first descriptor, the statistical modality is chosen from:
The fifth percentile minimum of the measured values of the signal on each electrode in the predefined area;
The ninety fifth percentile maximum of the measured values of the signal on each electrode of the predefined area;
The average of the measured values of the signal on each electrode of the predefined area;
The standard deviation of the measured values of the signal on each electrode of the predefined area;
The median of the measured values of the signal on each electrode of the predefined area; and
The interquartile of the measured values of the signal on each electrode of the predefined area.

This provision makes it possible to select a statistical modality that makes it possible to process the multiple measurements made while choosing the statistical modality that is relevant, according to whether it is wished to analyze maxima or minima for example. These modalities also make it possible to avoid extreme aberrant measurements.

According to one embodiment, the method of the present invention may be succeeded by a method for generating an electrophysiological parameter to characterize a cardiac activity.

Such a step may be implemented by a method for generating an electrophysiological parameter which comprises:
selection of a subset of electrophysiological descriptors from a set of predefined electrophysiological descriptors according to an input parameter defining a measurement context, each electrophysiological descriptor of the subset being associated with at least one channel, a signal type, a signal marker and a statistical modality for calculation;
arrangement of a plurality of surface electrodes on a patient's body;
recording of a plurality of cardiac electrical activities defining said channels, each channel being obtained by the recordings of at least two electrodes;
estimation of all the electrophysiological descriptors of the subset, each electrophysiological descriptor being calculated from the statistical modality that is applied to the signal marker of the acquired signal according to the signal type on a selected channel associated with said electrophysiological descriptor;
comparison of the value of all the electrophysiological descriptors with at least one threshold value specific to all the electrophysiological descriptors, said at least one threshold value being defined by a statistical distribution of said descriptors of a set of healthy patients;
calculation of a score defining an electrophysiological parameter as a function of the exceeding of at least one threshold value defined by the statistical distribution.

According to one aspect, the invention relates to a device or a system comprising means for implementing the method of the invention. The means may comprise calculators, memories, electronic boards, electrodes and electrode holders. The device or the system of the invention may comprise computers or servers when calculation resources are required. The invention is hereafter described such that the described characteristics may relate to the method of the invention or to the device or the system of the invention.

The invention also relates to a device for selecting a subset of electrophysiological descriptors. The device according to the invention is capable of implementing the method mentioned previously. Subsequently, the elements described in this description will be applicable both to the method according to the invention and to the device according to the invention.

The invention also relates to a device for selecting a subset of electrophysiological descriptors which comprises:
a plurality of surface electrodes configured to be deposited on a patient's body and to measure an electrical potential of the surface of the patient's body, each surface electrode defining a channel;
a means of measuring the signal of each channel;
a calculation means configured to:
   i. Estimate for a first set of patients not having a predefined condition, values of each electrophysiological descriptor of a set of first predefined descriptors, each first descriptor being associated with at least one channel, a signal type, a signal marker and a statistical modality for calculation; at least one first descriptor of the subset being associated with a statistical modality different from that of another first descriptor of the subset and a signal marker different from that of the other first descriptor;
   ii. Estimate for a second set of patients with the predefined condition, values of each descriptor of the set of predefined descriptors;
   iii. Generate a first vector characteristic of the condition of each patient of the first and second sets of patients in which each component corresponds to a condition relative to the predefined condition;
   iv. Generate a descriptor vector for each descriptor in a metric space in which each component corresponds to the value of the descriptor for each patient;
   v. Quantify for each descriptor a first proximity factor between the values of the components of the first characteristic vector and the values of the components of the descriptor vector;
   vᵢ. Select and include in the subset at least one descriptor having optimal quantified proximity factor values;
   vii. Quantify:
      a second proximity factor between the values of the components of each descriptor not selected during the first selection step and the values of the components of the descriptor vector(s) selected during the first selection step; and
      a third proximity factor between the values of the components of each descriptor not selected during the first selection step and the values of components of the characteristic vector;
   viii. Selecting and including in the subset at least one new descriptor as a function of the value of the second proximity factor and the value of the third proximity factor quantified during the second step of quantification.

One advantage of the invention is to propose a device for selecting a subset of different electrophysiological descriptors to characterize a cardiac activity. The selection device according to the invention makes it possible to select a subset of electrophysiological descriptors that is relevant to discriminate between two different populations, from a set of physiological descriptors. Thus, it is possible to select from a large number of descriptors that take into account different signal types, different signal markers and various statistical modalities for calculation, a relevant subset of descriptors. Furthermore, as the device according to the invention takes into account the third proximity factor, a set of electrophysiological descriptors that are not redundant with each other to characterize said cardiac activity is then obtained. The selection device according to the invention thus makes it possible to obtain an effective and non-redundant subset for the discrimination of two populations.

According to one embodiment, the electrodes are arranged on the surface of the patient using adhesive strips. This characteristic is a practical way of implementation to lay out the electrodes on the patient.

According to one embodiment, the device comprises a device for detecting a patient's breathing phases, preferably a plethysmography belt. This provision makes it possible to carry out measurements during a breathing phase of the patient that does not disturb said measurements.

According to one embodiment, the device according to the invention is capable of implementing the method according to the invention.

### BRIEF DESCRIPTION OF THE FIGURES

Other characteristics and advantages of the invention will become clearer upon reading the following detailed description, in reference to the appended figures, that illustrate:

FIG. 1: a schematic flowchart of the method according to the invention;

FIG. 2: a front view of the torso of a patient on which is arranged a plurality of measuring electrodes to implement a method according to the invention;

FIG. 3: a view of a plurality of measuring electrodes in a predefined area of the patient's body;

FIG. 4: two curves illustrating the method for calculating an asymmetry index;

FIG. 5: a graph illustrating the method for calculating a Kurtosis index;

FIG. 6: a view of a curve illustrating the method for calculating the number of areas of reduced amplitude.

### DESCRIPTION OF THE INVENTION

According to a first aspect, the invention relates to a method for selecting a subset of electrophysiological descriptors characteristic of a characteristic cardiac activity.

The invention also relates to a device for selecting a subset of electrophysiological descriptors characteristic of a characteristic cardiac activity. The device according to the invention is capable of implementing the invention mentioned above. Subsequently, the elements described in this description will be applicable both to the method according to the invention and to the device according to the invention.

The method according to the invention will be described in support of FIG. 1, which is a schematic flowchart of the method according to the invention.

According to a first aspect, the invention relates to a method for selecting a subset {Dₖ} of first electrophysiological descriptors Dᵢ characteristic of an electrical activity representative of a characteristic cardiac activity.

“Characteristic cardiac activity” is taken to mean a patient's cardiac activity which may be characteristic of said patient's condition.

The subset {Dₖ} of first electrophysiological descriptors Dᵢ is selected from a set {D_{N}} of electrophysiological descriptors. Thus, the method according to the invention relates to the selection, from a set {D_{N}} of electrophysiological descriptors, of a subset {Dₖ} of more restricted descriptors. The aim of the method according to the invention is to perform a relevant and reduced selection of several descriptors Dᵢ from the set {D_{N}}.

“Electrophysiological descriptor Dᵢ” is taken to mean a context of measurement of an electrical datum associated with an activity detected on the surface of a patient. Each first electrophysiological descriptor Dᵢ is associated with the recording of at least one channel Vᵢ. The recording of a channel Vᵢ corresponds to the recording of the electrical activity captured by at least one electrode EL arranged on the surface of a patient's body. For the purposes of the recordings, the invention may be implemented notably by means of a memory making it possible to record the data acquired and/or the data processed by one of the steps of the method according to the invention. Each first descriptor Dᵢ is associated with at least one channel Vᵢ on a predefined area Zᵢ on the patient's body. “Predefined area Zᵢ” is taken to mean the area of the patient's body on which are deposited the measuring electrode(s) EL, the electrical activities of which are recorded to obtain the channel(s) Vᵢ. Thus, the surface of a patient's body may be segmented into different functional and/or geometrical and/or physiological areas. These areas may therefore correspond to geographical areas on the patient's body, to functional areas in relation to the patient's cardiac activity, or to areas corresponding to the patient's physiology and/or physiology.

Each first descriptor Dᵢ is associated with a signal type Tᵢ measured on the selected channels Vᵢ. For example, signal type Tᵢ is taken to mean the measurement of a tension between two electrodes. The different types of signals Tᵢ that can be selected will be described later.

Each first descriptor Dᵢ is associated with a signal marker Mᵢ. “Signal marker Mᵢ” is taken to mean the characteristic of the signal type Tᵢ that will be measured. According to examples of used signal markers Mᵢ, certain comprise the measurement of frequency and energy characteristics of the signal. According to other examples, markers may comprise electrical voltage or tension measurement. The different signal markers Mᵢ that may be selected are described below.

Each first descriptor Dᵢ is associated with a statistical modality MSᵢ. “Statistical modality MSᵢ” is taken to mean a statistical measurement modality applied to the measurements made on the signals. For example, a statistical modality MSᵢ may be the calculation of the average of the signal marker Mᵢ measured on several electrodes. The different statistical modalities MSᵢ that may be selected will be described later.

Each first electrophysiological descriptor Dᵢ is therefore defined by both a selection of the predefined area Zᵢ of the patient's body, the signal type Tᵢ, the signal marker Mᵢ and the statistical modality MSᵢ.

According to one embodiment, at least two first descriptors Dᵢ, Dⱼ of the subset {Dₖ} are associated with a different statistical modality MSᵢ. In other words, two of the first descriptors Dᵢ, Dⱼ are not associated with the same statistical modality MSᵢ.

According to one embodiment, at least two first descriptors Dᵢ, Dⱼ of the subset {Dₖ} are associated with a different signal marker Mᵢ. In other words, two of the first descriptors Dᵢ, Dⱼ are not associated with the same signal marker Mᵢ.

A first step of the method according to the invention corresponds to an estimation EST₁, for a first set of patients ENS₁, of values of each first electrophysiological descriptor Dᵢ. The first set of patients only comprises patients not having a predefined condition ET₁. A predefined condition is a condition presenting particular characteristics at the cardiac level, regardless of whether the condition is pathological or not. According to one embodiment, the predefined condition ET₁ is a structural heart pathology. The estimation of the values of the first descriptors Dᵢ is performed for each first electrophysiological descriptor Dᵢ of the set of descriptors {D_{N}}.

A second step of the method according to the invention corresponds to an estimation EST₂, for a second set of patients ENS₂, of values of each first electrophysiological descriptor Dᵢ. The second set of patients only comprises patients with the predefined condition ET₁. The estimation of the values of the first descriptors Dᵢ is performed for each first electrophysiological descriptor Dᵢ of the set of descriptors {D_{N}}.

A next step is the generation GEN₁ of a first characteristic vector V₁ of the condition of each patient. This vector V₁ comprises one component per patient of the first set ENS₁ and one component per patient of the second set ENS₂. Each component of the characteristic vector V₁ corresponds to a condition relative to the predefined condition ET₁. According to one embodiment, the first vector comprises a first predefined value vp₁ for each patient of the first set ENS₁. Similarly, the first characteristic vector V₁ comprises a second predefined value vp₂ for each patient of the second set ENS₂. This provision makes it possible to have a characteristic vector V₁ which is representative of the condition of patients of the first and second sets ENS₁, ENS₂. According to an alternative, the components of the first characteristic vector V₁ are a function of the level of suffering from the predefined condition ET₁ of patients of the first and second sets ENS₁, ENS₂. According to this provision, the components of the first vector V₁ may, for example, have a low value when the patient is hardly affected by the predefined condition ET₁ and a high value when the patient is strongly affected by the predefined condition ET₁. According to one embodiment, the first characteristic vector V₁ comprises a component equal to “zero” for each patient of the first set ENS₁. According to one embodiment, the first characteristic vector Vᵢ comprises a component equal to “one” for each patient of the second set ENS₂.

A next step of the method according to the invention is the generation GEN₂ of a descriptor vector V_{d} for each first electrophysiological descriptor Dᵢ. This generation GEN₂ is performed in a metric space. Each component of the descriptor vector V_{d} corresponds to the value of said first electrophysiological descriptor Dᵢ. Thus, each descriptor vector V_{d} comprises one component per patient of the first and second sets ENS₁, ENS₂.

A next step of the method according to the invention relates to the first quantification QUAN₁ for each descriptor Dᵢ of a first proximity factor between the values of the components of the first characteristic vector V₁ and the values of the components of the descriptor vector V_{d}. This step makes it possible to estimate whether a correlation relationship exists between the values of the first characteristic vector V₁ and the values of the descriptor vector V_{d} for each descriptor Dᵢ considered.

According to one embodiment, the first quantification step QUAN₁ is carried out by quantifying for each descriptor Dᵢ the statistical correlation between the values of the components of the first characteristic vector V₁ and the values of the components of the descriptor vector V_{d}. Alternatively, this quantification may be performed by calculating a quadratic error.

According to one embodiment, the first quantification step QUAN₁ is performed using a method for selecting variables such as the LASSO method. Alternatively, a RIDGE regression may be used.

A next step of the method according to the invention is a first selection SEL₁ of at least one descriptor Dᵢ. In this step, at least one descriptor Dᵢ is selected as a function of the previously quantified values of the first proximity factor of each descriptor Dᵢ. The first descriptor(s) Dᵢ selected are included in the subset {Dₖ}. The selection is made by taking the descriptor(s) Dᵢ that comprise optimal proximity factor values from all the descriptors Dᵢ of the set {D_{N}}. Optimal value is taken to mean a value that reports the proximity of the descriptor vector V_{d} with the first vector V₁. In the event where the proximity factor is a correlation calculation, the optimal value is a maximum value. In a case where the proximity factor is a quadratic error, the optimal value is a minimum value. This step therefore makes it possible to select the descriptor(s) Dᵢ having the greatest proximity with the characteristic vector V₁.

A next step in the method is a step of second quantification QUAN₂. This step is broken down into two distinct steps. There is firstly the quantification of a second proximity factor between the components of the descriptor vectors V_{d} of the descriptors Dᵢ that were not selected at the first selection SEL₁ and the values of the components of the descriptor vector(s) V_{d} that were selected at the first selection step SEL₁. There is then the quantification of a third proximity factor between the components of the descriptor vectors V_{d} of the descriptors Dᵢ that were not selected during the first selection SEL₁ and the values of the components of the characteristic vector V₁. It may be noted that this step of second quantification may be performed from the values of the components of the descriptor vectors V_{d} and the characteristic vector or from a transformation in metric space of said components.

According to one embodiment, the step of second quantification QUAN₂ is carried out by quantifying the statistical correlation between the values of the components of the different vectors involved. Alternatively, this quantification may be performed by calculating a quadratic error.

According to one embodiment, the step of second quantification QUAN₂ is performed using a method for selecting variables such as the LASSO method. Alternatively, a RIDGE regression may be used.

A next step of the method according to the invention is a second selection step SEL₂ of at least one new descriptor Dᵢ and its inclusion in the subset {Dₖ}. The new descriptor Dᵢ is selected as a function of the values of the second proximity factor and the third proximity factor quantified during the second quantification QUAN₂. More precisely, during this selection step, the descriptor(s) Dᵢ having a high quantified proximity with the characteristic vector V₁ and a low quantified proximity with the descriptor vector(s) V_{d} previously selected are selected.

Taking into account the first proximity factor, the second proximity factor and the third proximity factor makes it possible to obtain a subset of descriptors Dᵢ which takes into account the redundancy of the information borne by several descriptors Dᵢ. According to these characteristics, a classification is obtained which no longer takes into account only the correlation between the descriptor vectors V_{d} and the first vector V₁, but which also makes it possible to select descriptor vectors V_{d} which have a high correlation value, but are not too similar to the first descriptor vectors V_{d} selected. This provision makes it possible to select a subset {Dₖ} that comprises non-redundant descriptors Dᵢ.

The method according to the invention makes it possible to select a subset {Dₖ} of descriptors Dᵢ which makes it possible to discriminate two sets of patients in an effective and non-redundant manner. The method according to the invention makes it possible to sort among a large number of electrophysiological descriptors Dᵢ to select therefrom a reduced number capable of differentiating patients according to their having or not the predefined condition ET₁.

According to one embodiment, the steps of second quantification QUAN₂ and second selection SEL₂ are reproduced from the descriptors Dᵢ not selected previously until a predefined number of descriptors Dᵢ is obtained. This method follows several iterations of the steps of second quantification QUAN₂ and second selection SEL₂ and thus makes it possible to obtain a subset {Dₖ} which comprises the desired number of electrophysiological descriptors Dᵢ.

According to one embodiment, the step of second quantification QUAN₂ comprises a step of projection in a first plane P₁ orthogonal to the descriptor vector(s) V_{d} associated with the last descriptors selected. During this projection step, all the descriptor vectors V_{d} that were not previously selected in the plane P₁ are projected. Similarly, the characteristic vector V₁ is projected in the plane P₁. This projection step may be assimilated to the step of second quantification QUAN₂ of the second proximity factor between the components of the descriptor vectors V_{d} not previously selected and the descriptor vectors V_{d} already included in the subset {Dₖ}. Indeed, the projection is equivalent to a quantification of the proximity factor, because the projection of a vector in the plane orthogonal to a vector that is very close to it gives a vector which is practically zero. According to this embodiment, the quantification of the third proximity factor is performed from the components of the projected descriptor vectors V_{d}. According to this embodiment, the quantification of the third proximity factor is performed from the components of the characteristic vector V₁ which has been projected. This vector orthogonalization method makes it possible to obtain a subset {Dₖ} in which each descriptor is relevant to discriminate between the two sets of patients ENS₁, ENS₂. The orthogonalization advantageously makes it possible not to select several electrophysiological descriptors Dᵢ which bear information that is redundant with that of the previously selected descriptors Dᵢ.

According to one embodiment, the predefined number of selected descriptors Dᵢ is determined by testing the effectiveness of the subset {Dₖ} of descriptors Dᵢ to discriminate the first set ENS₁ of patients from the second set ENS₂ of patients. In this way, the number of descriptors Dᵢ is searched for, which makes it possible to obtain the best performance for the subset {Dₖ}.

According to one embodiment, the predefined number of selected descriptors Dᵢ is determined by testing the effectiveness of a subset of descriptors by first testing the effectiveness of the first selected descriptor Dᵢ. Then, the effectiveness of the first two selected descriptors Dᵢ is tested. A number is then incremented in steps of “1”, testing the first three, then the first four descriptors. This iteration is continued until a number “n” of tested descriptors is reached. Finally, the predefined number of descriptors Dᵢ for which the discrimination performance of the first set ENS₁ of patients and the second set ENS₂ of patients is the highest is maintained. Alternatively, it is possible to start directly from a number “n” of descriptors and increment by steps of “1” until obtaining “N” descriptors. For example, the iterative process may be started by testing the effectiveness of a subset {Dₖ} of seven descriptors Dᵢ up to a set of twenty descriptors Dᵢ. For example, it may also start with the test with a subset of ten descriptors Dᵢ and end with a subset of thirty descriptors Dᵢ.

Electrophysiological Descriptors

As described previously, each first electrophysiological descriptor Dᵢ is associated with at least one channel Vᵢ of a predefined area Zᵢ of the patient's body, a signal type Tᵢ, a signal marker Mᵢ, and a statistical modality for calculation MSᵢ. These four elements will be described in detail below. To select a descriptor Dᵢ, a selection is made of a predefined area Zᵢ from a set of predefined areas Zᵢ. A selection is made of a signal type Tᵢ from a set of signal types Tᵢ. A selection is made of a signal marker Mᵢ from a set of signal markers Mᵢ. A selection is made of a statistical modality MSᵢ from a set of statistical modalities MSᵢ.

Patient's Body Area

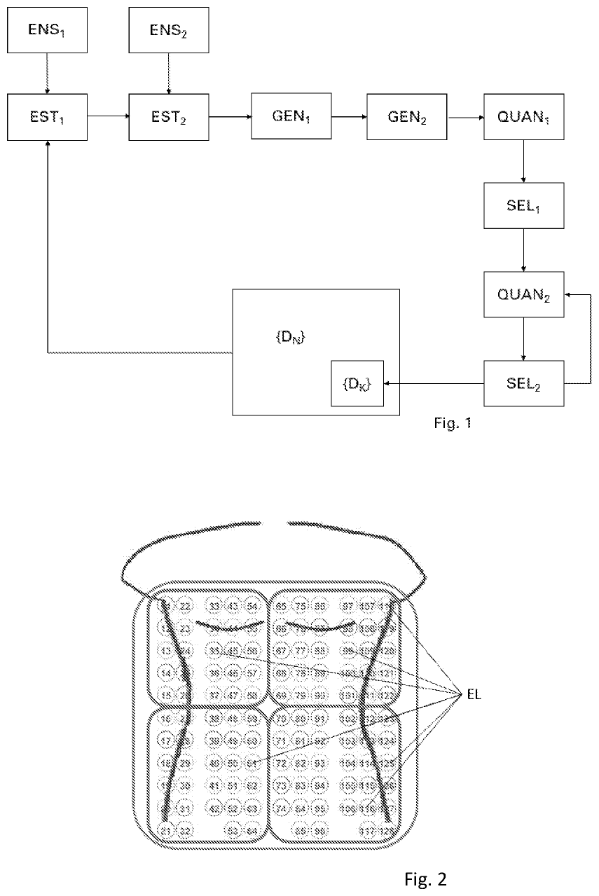

As shown in FIG. 2, a plurality of electrodes EL is deposited on the surface of the torso of each patient of the sets ENS₁ and ENS₂ to implement the selection method according to the invention. The number of electrodes in the example shown may vary, for example the number of electrodes may be much lower than that shown, or much higher. The plurality of electrodes EL covers a large part of the patient's torso. As can be seen, this plurality of electrodes is separated into four distinct areas on it. A first part of the electrodes EL is located on an upper right area Z₁ of the patient's torso. A second part of the electrodes EL is located on an upper left area Z₂ of the patient's torso. A third part of the electrodes EL is located on a lower right area Z₃ of the patient's torso. A fourth part of the electrodes EL is located on a lower left area Z₄ of the patient's torso. Finally, the plurality of electrodes EL is comprised in an area comprising the totality Z₅ of the patient's torso.

The demarcation between the areas situated to the left of the torso and those situated to the right of the torso is a vertical line passing through the center of the torso, or substantially through the center of the torso.

The demarcation between the areas situated on the lower torso and those situated on the upper torso is a horizontal line through the center of the torso.

Each area comprises a predefined number of electrodes EL. The number of electrodes arranged per area may be of the order of thirty or so. For example, there may be thirty electrodes EL per area.

Advantageously, each area comprises the same number of electrodes EL. Obviously, the area comprising the entire torso Z₅ comprises a different number of electrodes than the others, because this area Z₅ comprises the reunion of the electrodes of all the other areas Z₁, Z₂, Z₃ and Z₄.

Hereafter, when mention is made of the selection of electrodes in an area, the selection of one or more electrodes EL in said area will be meant.

Alternatively, a lower number of electrodes EL may be provided, for example nine electrodes EL per area Z₁, Z₂, Z₃, and Z₄. Thus, in this case, the area Z₅ comprising the entire torso of the patient comprises thirty-six electrodes EL.

Each channel Vᵢ is obtained by recording the electrical activity of at least two electrodes EL. These at least two electrodes may be two electrodes from one or more areas of the patient's torso. These at least two electrodes may also be an electrode from an area of the patient's torso and a reference electrode.

Geographical Groups

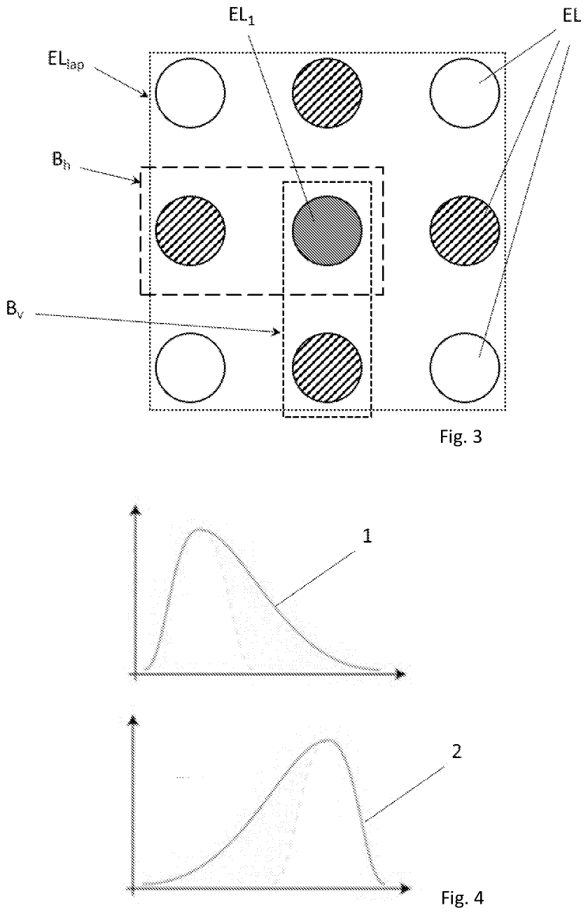

According to one embodiment, the set {D_{N}} comprises at least one second geographical electrophysiological descriptor Dᵢ. The at least one geographical descriptor Dᵢ is associated with at least one channel Vᵢ and with several geographical groups. A geographical group is formed by an electrode EL and the four electrodes EL that are situated directly next to it. A geographical group is shown in FIG. 3. This geographical group comprises a central electrode EL₁ and the electrode situated directly above it. It also comprises the electrode situated directly below the central electrode EL₁, the electrode situated directly to the left of the central electrode EL₁, and the electrode situated directly to the right of the central electrode EL₁. These four electrodes are shown hatched in FIG. 3. For the second geographical descriptors, the measurement of the value according to the signal type and the signal marker is performed on all the available geographical groups of the plurality of electrodes arranged on the patient's body. Other provisions of electrodes may be envisaged. It is notably possible to select the central electrode and the four electrodes situated at the top left, top right, bottom left and bottom right of the central electrode EL₁. These are the electrodes that appear without a pattern in FIG. 3. More electrodes may also be provided, for example nine electrodes in the geographical group. These are the nine electrodes EL in FIG. 3 for example.

For each geographical group, the value obtained for each electrode EL of said group is compared with at least one geographical threshold value. The at least one geographical threshold value is obtained from a statistical distribution of the value of the considered electrode EL of the set of patients ENS₁ not having the predefined condition ET₁. When at least three electrode values of a geographical group exceed their geographical threshold value, then the geographical group is considered as significant. Finally, the value of the descriptor is the number of significant geographical groups counted. Alternatively, a geographical group may be considered as signifying from two electrodes exceeding their geographical threshold value, or instead with four electrodes.

It may be noted that in the case of a second geographical descriptor, the statistical modality is not taken into account, the value of the descriptor being the number of geographical groups detected.

Advantageously, the subset {Dₖ} of descriptors comprises at least one first descriptor Dᵢ and at least one second geographical descriptor. Additionally, the subset {Dₖ} comprises several first descriptors Dᵢ. According to this alternative, the subset {Dₖ} comprises a second geographical descriptor per signal marker used in the first descriptors Dᵢ of the subset {Dₖ}.

Signal Type

Each electrophysiological descriptor Dᵢ is associated with a signal type Tᵢ. In this section, reference will be made to FIG. 3, which is a schematic representation of nine contiguous electrodes EL on the patient's body. In this figure, each circle represents an electrode EL. The dotted areas represent the different electrodes EL selected in the different signal types.

The signal type Tᵢ may preferably be chosen between four different signal types.

A first signal type Tᵢ is a unipolar signal. A unipolar signal is a signal taken between an electrode EL of the predefined area Zᵢ and a reference electrode. In other words, the unipolar signal type is the tension measured between the electrode of the predefined area and the reference electrode. “Reference electrode” is taken to mean an electrode that is not situated in one of the areas of the patient's torso defined previously. For example, a reference electrode may be an electrode placed on a lower limb or an upper limb of a patient.

A second signal type Tᵢ is a vertical bipolar signal. A vertical bipolar signal is a signal taken between an electrode in the predefined area and the electrode situated directly below it on the patient's torso. In other words, the signal type acquired is the tension between the two electrodes EL. A vertical bipolar signal is taken between two electrodes of the predefined area Zᵢ. These two electrodes form a vertical bipole Bᵥ.

A third signal type Tᵢ is a horizontal bipolar signal. A horizontal bipolar signal is a signal taken between an electrode in the predefined area and an electrode situated directly next to it along a horizontal line on the patient's torso. In other words, the signal type acquired is the tension between the two electrodes EL. The horizontal bipolar signal is taken between two electrodes of the predefined area Zᵢ. These two electrodes form a horizontal bipole Bₕ.

A fourth signal type Tᵢ is a Laplacian signal. A Laplacian signal is estimated by subtracting from the potential of a central electrode EL₁ the average of the potentials of the eight electrodes that are directly near said central electrode. In other words, the Laplacian signal is a tension composed between the central electrode EL₁ and a set of electrodes EL peripheral to the central electrode EL₁. These nine electrodes form a Laplacian electrode ELₗₐₚ.

Signal Markers

Each electrophysiological descriptor Dᵢ is associated with a signal marker Mᵢ. A signal marker Mᵢ is a modality of measuring a physical quantity associated with the types of signals measured by the electrodes EL.

The signal marker Mᵢ associated with a descriptor Dᵢ is preferably chosen from fourteen signal markers Mᵢ. These signal markers Mᵢ are described below.

A first signal marker Mᵢ corresponds to the measurement of an averaged electrical signal. Averaged electrical signal is taken to mean the calculation, made on the measured tension, of the average between the maximum peak and the minimum peak of the QRS. This measurement of QRS duration is generally quite representative of a cardiac activity.

Two signal markers which are measured on the filtered signal between 40 and 150 Hertz will now be described. The signal is filtered using a bandpass filter. Advantageously, the bandpass filter is a bidirectional Butterworth filter. A bi-directional Butterworth filter has the advantage of limiting oscillations due to filtering, which makes the calculation of values for some signal markers Mᵢ more accurate.

A signal marker Mᵢ on the filtered signal is the QRS duration on the filtered signal. To measure this value, a mark is placed at the start of the QRS and a second mark is placed at the end of the QRS. The time between the two marks is measured. This operation may be performed automatically thanks to a QRS start and end detection algorithm. Alternatively, this time can be measured manually by an operator on an interface. An automatic measurement of the QRS duration and a manual check of said measurement by the operator on the interface may also be provided.

According to one embodiment, the QRS duration is detected by moving a sliding window measuring the energy of the filtered signal. When an energy threshold is exceeded, a mark is placed that marks the start of the window. The QRS end mark is placed in the same way.

Another signal marker Mᵢ is the measurement of the fragmentation of the filtered averaged signal between 40 Hertz and 250 Hertz. According to this marker Mᵢ, the number of QRS peaks on the filtered signal is measured. Peak is taken to mean a local maximum of the filtered signal curve. The number of peaks is measured on the section of the curve corresponding to the QRS. The start and end marks of the QRS are set in the same way as for the previous marker Mᵢ, which as a reminder is the QRS duration marker on the filtered signal.

The following markers Mᵢ are calculated on the decomposition into wavelets of the signal. For these markers Mᵢ, either continuous wavelet decomposition or discrete wavelet decomposition may be used.

The four markers Mᵢ shown below are calculated on the discrete wavelet decomposition.

A first marker Mᵢ is the calculation of the energy on the discrete wavelet decomposition of the signal. Specifically, the energy is calculated on the sum of the coefficients over several levels. Typically, the sum of the coefficients is made between 64 Hertz and 1024 Hertz, i.e. on the four levels of this frequency band. According to one embodiment, the measured energy is normalized with respect to the QRS duration. Alternatively or additionally, the energy is normalized with respect to the maximum signal amplitude.

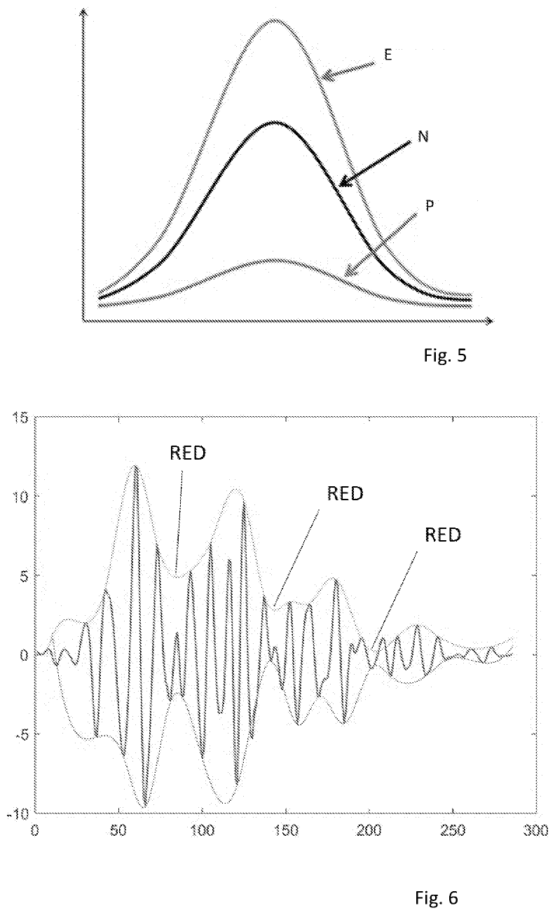

A second marker Mᵢ calculated on the discrete wavelet transform is the measurement of the so-called Kurtosis index Sₖᵤ. “Kurtosis” is taken to mean an index making it possible to estimate the spread of a given curve. FIG. 5 illustrates several measurements of curve spread on three exemplary curves. For a flat curve P, the Kurtosis index is negative. For a slender curve E, the index is positive. Thus, the more the curve is spread, the more negative the Kurtosis. When the curve is narrow, the Kurtosis is positive. The Kurtosis of a curve representing a normal distribution N is equal to zero. Specifically, the Kurtosis Sₖᵤ is calculated on the sum of the coefficients over several levels of the discrete wavelet decomposition. Typically, the sum of the coefficients is made between 64 Hertz and 1024 Hertz, i.e. on the four levels of this frequency band.

A third marker Mᵢ calculated on the discrete wavelet transform is the measurement of the Fischer asymmetry coefficient. This coefficient may also be called “Skewness”. This coefficient makes it possible to estimate the asymmetry of a given curve. FIG. 4 illustrates two asymmetry measurements on two curves given as examples. Curve 1 is a curve tending to the left and curve 2 is a curve tending to the right. The Fischer asymmetry coefficient has a positive value when the curve tends to the left. This is the case for curve 1. The Fischer asymmetry coefficient has a negative value when the curve tends to the right. This is the case in FIG. 2. Specifically, the Fischer asymmetry coefficient is calculated on the sum of the coefficients over several levels of the discrete wavelet decomposition. Typically, the sum of the coefficients is made between 64 Hertz and 1024 Hertz, i.e. on the four levels of this frequency band.

A fourth marker Mᵢ calculated on the discrete wavelet transform is the measurement of the number of local minima chains of said decomposition. Specifically, a local minima chain is the presence on several discrete wavelet decomposition levels of a same minimum. By measuring the number of minima that are found in each decomposition level, the number of local minima chains is measured. Typically, the measurement is made between 64 Hertz and 1024 Hertz, i.e. on the four levels of this frequency band. The minima repeating in the 64 Hertz to 128 Hertz bands, then 128 Hertz to 256 Hertz, then 256 Hertz to 512 Hertz and finally 512 Hertz to 1024 Hertz are therefore sought. Alternatively, the measurement of the number of local minima chains may be carried out on the continuous wavelet decomposition.

Another signal marker Mᵢ that may be chosen is the measurement on the continuous wavelet decomposition of the number of local maxima chains. In concrete terms, a local maxima chain is the presence on several discrete wavelet decomposition levels of a same maximum. By measuring the number of maxima that are found in each decomposition level, the number of local maxima chains is measured. Typically, the measurement is made between 64 Hertz and 1024 Hertz, i.e. on the four levels of this frequency band. The maxima repeating in the 64 Hertz to 128 Hertz bands, then 128 Hertz to 256 Hertz, then 256 Hertz to 512 Hertz and finally 512 Hertz to 1024 Hertz are therefore sought. Alternatively, the number of local maxima chains may be measured on the discrete wavelet decomposition.

The following two signal makers Mᵢ are measured on the wavelet of the signal comprised in the frequency band ranging from 256 Hertz to 512 Hertz of the signal. The first concerns the measurement of the Kurtosis index Sₖᵤ on this wavelet. “Kurtosis Sₖᵤ” is taken to mean the same indicator as described above in the application. The second signal marker Mᵢ measured on this wavelet is the measurement of the number of reduced amplitude areas RED of the wavelet. To calculate the number of reduced amplitude areas, the upper and lower signal envelopes are created. Thus, the number of areas of reduced amplitude is calculated on the signal envelopes. This is illustrated by FIG. 6 which shows a signal and the reduced amplitude areas RED detected.

The following three signal markers Mᵢ are measured on the wavelet of the signal comprised in the frequency band ranging from 128 Hertz to 256 Hertz of the signal. The first concerns the measurement of the Kurtosis index Sₖᵤ on this wavelet. Kurtosis Sₖᵤ is taken to mean the same indicator as that described previously in the application. The second signal marker Mᵢ measured on this wavelet is the measurement of the number of areas of reduced amplitude of the wavelet. The number of areas of reduced amplitude is calculated in the same way as for the marker concerning the wavelet of the signal in the frequency range 256 Hertz to 512 Hertz of the signal. The third signal marker concerns the measurement of the RMS of the wavelet in the frequency band 128 Hertz to 256 Hertz. RMS, or Root Mean Square, is taken to mean the measurement of the effective amplitude of the signal.

Finally, a signal marker Mᵢ that may be selected is measured on the wavelet of the signal comprised in the frequency band ranging from 64 to 128 Hertz. This signal marker Mᵢ relates to the measurement of the RMS (Root Mean Square) effective amplitude of the signal.

Other signal markers Mᵢ may be used beyond the fourteen signal markers Mᵢ described. For example, signal markers Mᵢ may be used which are combinations of signal markers Mᵢ already described.

Statistical Modality

Each first electrophysiological descriptor Dᵢ is associated with a statistical modality MSᵢ. Statistical modality MSᵢ is taken to mean a modality for processing the different quantities measured in order to calculate a value for each descriptor Dᵢ.

It may be recalled that for each first descriptor Dᵢ an area of the body of the patient Zᵢ is selected, a signal type Tᵢ (therefore the way in which the individual signals of each electrode EL are captured and used), and a signal marker Mᵢ. It should be specified that once these three choices have been made, the signal marker Mᵢ for the selected signal type is measured for each electrode of the body area of the patient Zᵢ available in said selected area Zᵢ. Thus, for each descriptor Dᵢ, a plurality of measurements of the value of the signal marker Mᵢ is obtained. Using the statistical modality MSᵢ makes it possible to transform this plurality of values into a final value for the selected descriptor Dᵢ.

For each descriptor Dᵢ, a statistical modality MSᵢ may be selected from a set of available statistical modalities MSᵢ.

A first statistical modality is the fifth percentile minimum of the measured values. It will be recalled that, for the values, all the values measured on each electrode in the predefined area of the body of patient Zᵢ are taken. For this statistical modality, the five percent of the lowest values are removed from all the measured values and the minimum value is selected from the remaining values. This statistical modality has the advantage, by removing the five percent of the lowest values, of eliminating outliers that could distort the representativeness of the measurement.

A second statistical modality that can be selected is the 95^{th} percentile maximum. For this statistical modality, the five percent of the highest values are taken from all the selected values. The highest value of the remaining values is then selected. This statistical modality makes it possible not to take into account, for a measurement of a maximum value, the outliers which could appear in the highest values measured. In this way, there is an upper limit representative of all the measured values.

A third statistical modality MSᵢ is the average of the measured values. The average is a conventional indicator and representative of a distribution.

A fourth statistical modality MSᵢ is the standard deviation calculated on the set of measured values. The standard deviation is a representative value of the dispersion of the values. In our case, the dispersion may be a significant value, a large variance in the measurements being able to be the sign of a disorder in the patient's cardiac activity.

A fifth statistical modality MSᵢ that may be selected is the median. The median value of a set of values is the value making it possible to separate all of the values into two sets of the same size. This value provides a teaching which may vary from that given by the average value, because the median makes it possible not to give too much importance to outliers close to the maximum and minimum of the values measured.

A sixth statistical modality MSᵢ is the value of the interquartile. To calculate this value, the value of the 25^{th} percentile and the value of the 75^{th} percentile are calculated. The value of the interquartile represents the difference between the value of the 75^{th} percentile and the value of the 25^{th} percentile. The interquartile is an interesting statistical value to look at to characterize the distribution of the measured values.

Device for Measuring Descriptor Values

The invention also relates to a device for measuring the values of the electrophysiological descriptors previously described. The invention also relates to the means used to implement the method for selecting a subset of descriptors according to the invention. The characteristics described above concerning the method according to the invention also apply to the device according to the invention. The characteristics described below for the device also apply to the method according to the invention.

The device for measuring the values of electrophysiological descriptors comprises a plurality of electrodes which are arranged on the surface of the patient's body. Each surface electrode defines a channel Vᵢ.

According to one embodiment, the device comprises adhesive strips comprising the surface electrodes. According to this aspect, the adhesive strips are intended to be applied to the surface of the patient's body. Advantageously, each adhesive strip comprises several surface electrodes. This provision makes it easier to install the electrodes on the patient, the installation of a strip comprising several electrodes being simpler than installing the electrodes one by one.

According to one aspect, the device comprises a vest or jacket comprising the plurality of measuring electrodes EL. The vest is intended to be put on by the patient. This provision enables a rapid installation of the device on the patient. According to one example, the device comprises at least 14 electrodes.

The device comprises a means of measuring the signal of each channel Vᵢ. More precisely, the measurement means is configured to measure an electrical potential of each of the channels Vᵢ. For example, the measurement means may be an acquisition card. The acquisition card may comprise an input to collect an electrical signal, and an analog digital converter to digitize the acquired signal. The digitized signal is then transmitted to a calculator. For example, the digitized signal may be transmitted to a computer that performs the processing steps on the signal.

The device comprises a means of calculation. The calculation means records the measurements of channels Vᵢ supplied by the measurement means. The calculator then processes this data.

The calculator calculates the value of each descriptor Dᵢ of the set {Dₖ} for each patient of each set ENS₁ and ENS₂. This calculation is performed from measurements of channels Vᵢ. The calculation is performed according to the predefined area Zᵢ, signal type Tᵢ, signal marker MSᵢ and statistical modality STᵢ selected for the descriptor Dᵢ in question.

The calculator then performs the steps of generation GEN₁ of the first vector Vᵢ as described previously. It also performs the steps of generation GEN₂ of the descriptor vector V_{d}, quantification QUAN of the correlation, first classification CLAS₁, second classification CLAS₂, and selection SEL described in the method according to the invention. It is able to perform all the other calculation steps described in this application.

According to one embodiment, the device comprises a device for detecting the breathing phases of the patient. Such a device detects when the patient is in the expiration phase or “flat” breathing phase. It also detects when the patient is in the inspiration phase. Breathing tends to interfere with the measurements carried out at the level of the electrodes EL. This is notably the case during inspiration phases during which heart beats and their measurement may be affected. Preferably, the measurement of the potential of each channel Vᵢ is performed during the expiration phase. This provision makes it possible to avoid disturbances caused by a measurement during inhalation phases. The device for detecting breathing phases may be connected to the calculator. Alternatively, it is connected to the means of measuring the signal of each channel Vᵢ.

According to one embodiment, the device for detecting breathing phases is a plethysmography belt. The plethysmography belt is a practical way to perform this type of detection.

Nomenclature:
Dᵢ: Electrophysiological descriptor
{Dₖ}: Subset of electrophysiological descriptors
{D_{N}}: Set of electrophysiological descriptors
Inp: Input parameter
Vᵢ: Channel
Zᵢ: Predefined area of the patient's body
Z₁: Upper right area of the patient's torso
Z₂: Upper left area of the patient's torso
Z₃: Lower right area of the patient's torso
Z₄: Lower left area of the patient's torso
Z₅: Area covering the totality of the patient's torso
Tᵢ: Signal type
Mᵢ: Signal marker
MSᵢ: Statistical modality for calculation
EL: Electrode
EL₁: Central electrode
ELₗₐₚ: Laplacian electrode
Bᵥ: Vertical bipole
Bₕ: Horizontal bipole
DISPO: arrangement of a plurality of electrodes
ENR: Recording of a plurality of electrical activities
EST: Estimation of descriptors
COMP: Comparison of the value of a descriptor with a threshold value
V_{threshold}: Threshold value
CALC: Calculation of a score
Sₖᵤ: Kurtosis
N: Curve representing a normal distribution
P: Flat curve
E: Slender curve
1: Curve tending to the left
2: Curve tending to the right
RED: Reduced amplitude range

1. A method implemented by computer for selecting a subset of first electrophysiological descriptors characteristic of a characteristic cardiac electrical activity from a set of first predefined descriptors wherein each first electrophysiological descriptor is associated with at least one channel, a signal type, a signal marker and a statistical modality for calculation; at least one first descriptor of the subset being associated with a statistical modality different from that of another first descriptor of the subset and with a signal marker different from that of the other first descriptor; the method comprising recording a plurality of electrical activities defining said channels and:
estimating, for a first set of patients not having a predefined condition, of values of each descriptor of the set of predefined descriptors;
estimating, for a second set of patients having the predefined condition, of values of each descriptor of the set of predefined descriptors;
generating a first vector characteristic of the condition of each patient of the first and second sets of patients in which each component corresponds to a condition relative to the predefined condition;
generating a descriptor vector for each descriptor in a metric space in which each component corresponds to the value of the descriptor for each patient;
first quantifying for each descriptor a first proximity factor between the values of the components of the first characteristic vector and the values of the components of the descriptor vector;
first selection and inclusion in the subset of at least one descriptor having optimal quantified proximity factor values;
second quantifying:
of a second proximity factor between the values of the components of each descriptor not selected during the first selection step and the values of the components of the descriptor vector selected during the first selection step; and
of a third proximity factor between the values of the components of each descriptor not selected during the first selection step and the values of the components of the characteristic vector;
second selecting and inclusion in the subset of at least one new descriptor as a function of the value of the second proximity factor and the value of the third proximity factor quantified during the step of second quantification.

2. The method according to claim 1, wherein the steps of second quantification and second selection are reproduced from the descriptors not previously selected until a predefined number of selected descriptors is obtained.

3. The method according to claim 1, wherein the quantification of the first proximity factor and/or the second proximity factor, and/or the third proximity factor is a calculation of the correlation of vectors between them.

4. The method according to claim 1, wherein the step of second quantification comprises a step of projection, in a first plane orthogonal to the descriptor vector associated with the last descriptors selected, of each descriptor vector not selected and of the characteristic vector; wherein the quantification of the third proximity factor and performed from the projected vector projection components; and wherein the second selection is performed as a function of the third proximity factor only.

5. The method according to claim 2 wherein the predefined number of selected descriptors is determined by testing the effectiveness of a set of first n descriptors selected to characterize a given electrical activity, n corresponding to the number of descriptors taken from the selected descriptors, starting from the first to the nth descriptor selected, and incrementing the value of n by steps of 1.

6. The method according to claim 1, wherein the set comprises at least one second geographical descriptor associated with several channels and several geographical groups, each geographical group being formed by a central channel and the at least four channels close to the central channel, the value of the electrophysiological descriptor being determined:
by comparing the value, for each geographical group, of the measurement of each channel according to the signal type and the signal marker selected with at least one geographical threshold value specific to said electrophysiological descriptor and said channel; and
by counting the number of geographical groups for which the value of at least three channels exceeds its own geographical threshold value.

7. The method according to claim 1, wherein for each first descriptor, the at least one channel is derived from a predefined area on the body of the patient is chosen between:
an upper right area of the torso;
an upper left area of the torso;
a lower right area of the torso;
a lower left area of the torso; and
an entire torso of the patient.

8. The method according to claim 1, wherein for each descriptor, the signal type analyzed is chosen between:
a unipolar signal taken between an electrode of the chosen body area and a reference electrode;
a vertical bipolar signal taken between two electrodes of the given area, one of the two electrodes being offset along a vertical line with respect to the other electrode;
a horizontal bipolar signal taken between two electrodes of the given area, one of the two electrodes being offset along a horizontal line with respect to the other electrode; and
a Laplacian signal estimated by subtracting from the potential of a central electrode the average tension of the eight electrodes directly adjacent to said central electrode.

9. The method according to claim 1, wherein for at least one descriptor, the signal marker is the measurement of the voltage of an averaged signal.

10. The method according to claim 1, wherein for at least one descriptor, the signal marker is the measurement, on the averaged and filtered signal between 40 and 250 Hertz, of the duration of depolarization of the ventricles or fragmentation of the signal during depolarization of the ventricles.

11. The method according to claim 1, wherein for at least one descriptor, the signal marker is the measurement on the discrete wavelet decomposition of the signal:
of the energy of the sum of the wavelets;
of the Kurtosis;
of the Fisher asymmetry coefficient; or
of the number of local minima.

12. The method according to claim 1, wherein for at least one descriptor, the signal marker is the measurement on the continuous wavelet decomposition of the signal of the number of chains of local maxima.

13. The method according to claim 1, wherein for at least one descriptor, the signal marker is:
either the measurement on the wavelet taken between 256 and 512 Hertz of the signal:
i. of the Kurtosis; or
ii. of the number of areas with reduced amplitudes;
or the measurement on the wavelet taken between 128 and 256 Hertz of the signal:
iii. of the Kurtosis;
iv. of the number of areas of reduced amplitude; or
v. of the RMS;
or the measurement on the wavelet taken between 64 and 128 Hertz of the RMS (Root Mean Square).

14. The method according to claim 1, wherein for each first descriptor, the statistical modality is chosen from:
the fifth percentile minimum of the measured values of the signal on each electrode of the predefined area;
the ninety fifth percentile maximum of the measured values of the signal on each electrode of the predefined area;
the average of the measured values of the signal on each electrode of the predefined area;
the standard deviation of the measured values of the signal on each electrode of the predefined area;
the median of the measured values of the signal on each electrode of the predefined area; and
the interquartile of the measured values of the signal on each electrode of the predefined area.

15. A device for selecting a subset of electrophysiological descriptors, the device comprising:
a plurality of surface electrodes configured to be deposited on a patient's body and to measure an electrical potential of the surface of the patient's body, each surface electrode defining a channel;
a means of measuring the signal of each channel;
at least one calculation means configured to:
i. estimate for a first set of patients not having a predefined condition, the values of each electrophysiological descriptor of a set of first predefined descriptors, each first descriptor being associated with at least one channel, a signal type, a signal marker and a statistical modality for calculation; at least one first descriptor of the subset being associated with a statistical modality different from that of another first descriptor of the subset and a signal marker different from that of the other first descriptor;
ii. estimate for a second set of patients having the predefined condition the values of each descriptor of the set of predefined descriptors;
iii. generate a first vector characteristic of the condition of each patient of the first and second sets of patients in which each component corresponds to a condition relative to the predefined condition;
iv. generate a descriptor vector for each descriptor in a metric space in which each component corresponds to the value of the descriptor for each patient;
v. quantify for each descriptor a first proximity factor between the values of the components of the first characteristic vector and the values of the components of the descriptor vector;
vᵢ. select and include in the subset at least one descriptor having optimal quantified proximity factor values;
vii. quantify:
a second proximity factor between the values of the components of each descriptor not selected during the first selection step and the values of the components of the descriptor vector selected during the first selection step; and
a third proximity factor between the values of the components of each descriptor not selected during the first selection step and the values of the components of the characteristic vector;
viii. select and include in the subset at least one new descriptor as a function of the value of the second proximity factor and the value of the third proximity factor quantified during the step of second quantification.

16. A device for selecting a subset of electrophysiological descriptors comprising a plurality of electrodes, a receiver of the signals measured by the electrodes, a memory for recording the measured data and a calculator for performing operations and processing on the measured data, said device comprising means configured to implement the method of claim 1.
